(19) United States
(12) Patent Application Publication
(10) Pub. No.: US 2011/0039933 A1
(43) Pub. Date: Feb. 17, 2011

(54) S1P-1 RECEPTOR AGONISTS

(75) Inventors: Ghotas Evindar, Waltham, MA (US); Hongfeng Deng, Acton, MA (US)

Correspondence Address: GLAXOSMITHKLINE;GLOBAL PATENTS, FIVE MOORE DR., PO BOX 13398, MAIL STOP: C.2111F, RESEARCH TRIANGLE PARK, NC, 27709-3398, US

(73) Assignee: PRAECIS PHARMACEUTICALS INCORPORATION, Waltham, MA (US)

(21) Appl. No.: 12/438,063

(22) PCT Filed: Aug. 7, 2007
(86) PCT No.: PCT/US07/17542

§ 371 Date: Mar. 26, 2009

Related U.S. Application Data
(60) Provisional application No. 60/823,403, filed on Aug. 24, 2006.
(60) Provisional application No. 60/827,948, filed on Oct. 3, 2006.

(51) Int. Cl. A61K 31/197; C07C 229/42; A61P 43/00; C07C 227/00

(52) U.S. Cl. 514/563; 562/448; 562/450

(57) ABSTRACT

The invention provides compounds formula (I), their preparation, and their use as pharmaceutically active immuno-suppressive agents for the treatment of autoimmune disorders, organ transplant rejection, disorders associated with an activated immune system, as well as other disorders modulated by lymphopenia or SIP receptors.

### RELATED APPLICATIONS

This application is related and claims priority to U.S. Provisional Application Ser. No. 60/823,403, filed Aug. 24, 2006 and U.S. Provisional Application Ser. No. 60/827,948, filed Oct. 3, 2006. The entire contents of which are incorporated herein by this reference.

### BACKGROUND OF THE INVENTION

The sphingosine-1-phosphate (S1P) receptors 1-5 constitute a family of seven transmembrane G-protein coupled receptors. These receptors, referred to as S1P1 to S1P5, are activated via binding by sphingosine-1-phosphate, which is produced by the sphingosine kinase-catalyzed phosphorylation of sphingosine. S1P receptors are cell surface receptors involved in a variety of cellular processes, including cell proliferation and differentiation, cell survival, cell invasion, lymphocyte trafficking, and cell migration. Sphingosine-1-phosphate is found in plasma and a variety of other tissues, and exerts autocrine and paracrine effects, including regulating the secretion of growth factors.

Administration of S1P to an animal results in sequestration of lymphocytes into the lymph nodes and Peyers patches without causing lymphocyte depletion. This activity, which is of potential utility in treating diseases or conditions associated with inappropriate immune response, including transplant rejection, autoimmune diseases, as well as other disorders modulated by lymphocyte trafficking, is believed to proceed via activation of the S1P1 receptor. Administration of SIP in vivo has been shown to cause hypotension and bradycardia, which are believed to be due to signaling through one or more of the other S1P receptors, i.e. S1P2 to S1P5. Accordingly, there is a need for compounds which are potent and selective agonists of the S1P-1 receptor.

### SUMMARY OF THE INVENTION

These and other needs are met by the present invention which is directed to a compound of formula I:

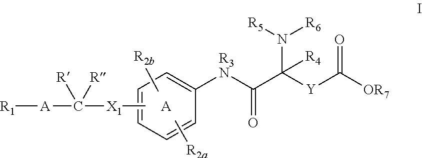

or a pharmaceutically acceptable salt thereof, wherein:

R₁ is hydrogen, halogen, cyano, alkyl, aryl, heteroaryl, cycloalkyl, heterocycloalkyl, aralkyl, heteroalkyl, —O-alkyl, —O-aryl, —O-heteroaryl, —S-alkyl, alkylene-O-alkyl, alkylene-CO₂H, alkylene-CO₂alkyl, alkylSO₂, alkylenesulfonyl, alkylene-CO-amino, alkylene-CO-alkylamino, alkylene-CO-dialkylamino, alkylene-NH—CO₂H, alkylene-NH—CO₂alkyl —CO₂alkyl, —OH, —C(O)-alkyl, —C(O)O-alkyl, —CONH₂, —CO-alkylamino, —CO-dialkylamino, amino, alkylamino, or dialkylamino, any of which may be optionally substituted on carbon with 1, 2, or 3 groups selected from halo, alkyl, OH, or —O-alkyl, which may itself be substituted on carbon with halo;

A is (C₁-C₂₀)alkylene, (C₂-C₂₀)alkenylene, or (C₂-C₂₀)alkynylene, each of which may be optionally substituted on carbon with 1, 2, or 3 groups selected from OH, CO₂H, CO₂alkyl, halogen, amino, alkylamino, dialkylamino, —O-alkyl, alkylene-O-alkyl, alkylene-OH, or alkylene-CO₂H;

X₁ is a bond or is CH₂, O, —CH₂O—, S, —S(O), —S(O)₂, —C(O)—, —C(O)O—, or NRₓ, wherein Rₓ is H or (C₁-C₆)alkyl;

R′ and R″ are each independently hydrogen, halogen, alkyl optionally substituted on carbon with halogen, alkyl, or taken together with the carbon to which they are attached form C═O or a 3, 4, 5, or 6-membered ring, optionally containing 1 or 2 heteroatoms selected from ONH, N-alkyl, SO, or SO₂, any of which may be optionally substituted on carbon with alkyl or halogen;

R₂ₐ is halogen, cyano, alkyl, aryl, heteroaryl, cycloalkyl, heterocycloalkyl, aralkyl, heteroalkyl, —O-alkyl, —O-aryl, —O-heteroaryl, aralkoxy, heteroaralkoxy, —S-alkyl, alkylene-O-alkyl, alkylene-CO₂H, alkylene-CO₂alkyl, alkylSO₂, alkylenesulfonyl, alkylene-CO-amino, alkylene-CO-alkylamino, alkylene-CO-dialkylamino, alkylene-NH—CO₂H, alkylene-NH—CO₂alkyl —CO₂alkyl, —OH, —C(O)-alkyl, —C(O)O-alkyl, —CONH₂, —CO-alkylamino, —CO-dialkylamino, amino, alkylamino, and dialkylamino, any of which may be optionally substituted on carbon with 1, 2, or 3 groups selected from halo, alkyl, OH, or —O-alkyl;

R_{2b} is hydrogen, halogen, cyano, alkyl, aryl, heteroaryl, cycloalkyl, heterocycloalkyl, aralkyl, heteroalkyl, —O-alkyl, —O-aryl, —O-heteroaryl, aralkoxy, heteroaralkoxy, —S-alkyl, alkylene-O-alkyl, alkylene-CO₂H, alkylene-CO₂alkyl, alkylSO₂, alkylenesulfonyl, alkylene-CO-amino, alkylene-CO-alkylamino, alkylene-CO-dialkylamino, alkylene-NH—CO₂H, alkylene-NH—CO₂alkyl —CO₂alkyl, —OH, —C(O)-alkyl, —C(O)O-alkyl, —CONH₂, —CO-alkylamino, —CO-dialkylamino, amino, alkylamino, and dialkylamino, any of which may be optionally substituted on carbon with 1, 2, or 3 groups selected from halo, alkyl, OH, or —O-alkyl;

R₃ is hydrogen, alkyl, or cycloalkyl, either of which may be optionally substituted on carbon with 1, 2, or 3 groups selected from halo, alkyl, OH, or —O-alkyl;

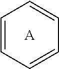

is phenyl or pyridyl;

Y is C₁-C₄ alkylene, optionally substituted on carbon with 1, 2, or 3 groups selected from halo, alkyl, OH, or —O-alkyl;

R₄ is hydrogen;

R₅ and R₆ are each independently selected from the group consisting of hydrogen, alkyl, alkylene-OH, aryl, alkylene-O-alkyl, —CO₂H, CO₂-alkyl, alkylene-OC(O)alkyl, cycloalkyl, heterocyclo, —C(O)-alkyl, —C(O)-aryl, C(O)-aralkyl, —C(O)—Oalkyl, —C(O)—Oaryl, —C(O)—Oaralkyl, alkylene-amino, alkylene-alkylamino, and alkylene-dialkylamino, any of which may be optionally substituted on carbon with halogen, alkyl, hydroxyl, CO₂H, CO₂alkyl or alkoxy; or

R₅ and R₆, together with the nitrogen to which they are attached, may form a 3, 4, 5, or 6-membered saturated or unsaturated ring, optionally containing 1 or 2 additional heteroatoms selected from O, S, NH, or N-alkyl, and optionally substituted on carbon with halogen, alkyl, hydroxyl, or alkoxy; R₅ and R₃ may form a 5, 6, 7, or 8-membered saturated or unsaturated ring, optionally containing 1 or 2 additional heteroatoms selected from O, S, NH, or N-alkyl, and optionally substituted on carbon with halogen, alkyl, hydroxyl, or alkoxy; and

R₇ is selected from the group consisting of hydrogen, alkyl, aryl or aralkyl.

The invention also provides a compound which is:

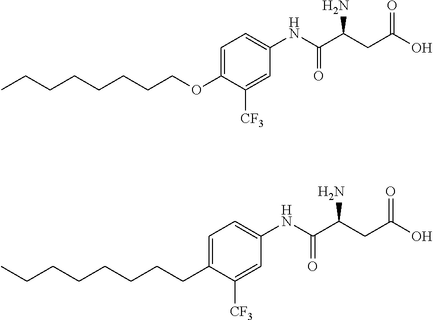

as well as pharmaceutically acceptable salts, phosphate derivatives, phosphate mimics, or phosphate precursor analogs thereof.

The invention is also directed to a method of treating an autoimmune disorder, comprising, administering to a subject in need thereof a pharmaceutically acceptable amount of a compound of the invention.

The invention is also directed to a method treating transplant rejection comprising, administering to a subject in need thereof a pharmaceutically acceptable amount of a compound of the invention.

The invention is also directed to a pharmaceutical composition, comprising a compound of the invention admixed with a pharmaceutically acceptable carrier.

The invention is also directed to a process for making a compound of the invention as provided herein.

### DETAILED DESCRIPTION OF THE INVENTION

### Definitions

The following definitions are used, unless otherwise described.

“Halogen” or “halo” means fluoro (F), chloro (Cl), bromo (Br), or iodo (I).

The term “hydrocarbon” used alone or as a suffix or prefix, refers to any structure comprising only carbon and hydrogen atoms up to 14 carbon atoms.

The term “hydrocarbon radical” or “hydrocarbyl” used alone or as a suffix or prefix, refers to any structure as a result of removing one or more hydrogens from a hydrocarbon.

The term “alkyl' used alone or as a suffix or prefix, refers to monovalent straight or branched chain hydrocarbon radicals comprising 1 to about 12 carbon atoms.

The term “alkylene” used alone or as suffix or prefix, refers to divalent straight or branched chain hydrocarbon radicals comprising 1 to about 12 carbon atoms, which serves to links two structures together.

The term “cycloalkyl” used alone or as suffix or prefix, refers to a saturated or partially unsaturated monovalent ring-containing hydrocarbon radical comprising at least 3 up to about 12 carbon atoms.

The term “aryl” used alone or as suffix or prefix, refers to a monovalent hydrocarbon radical having one or more polyunsaturated carbon rings having aromatic character, and comprising 5 up to about 14 carbon atoms.

The term “heterocycle” used alone or as a suffix or prefix, refers to a ring-containing structure or molecule having one or more multivalent heteroatoms, independently selected from N, O and S, as a part of the ring structure and including at least 3 and up to about 20 atoms in the ring(s). Heterocycle may be saturated or unsaturated, containing one or more double bonds, and heterocycle may contain more than one ring. When a heterocycle contains more than one ring, the rings may be fused or unfused. Fused rings generally refer to at least two rings share two atoms therebetween. Heterocycle may have aromatic character or may not have aromatic character.

The terms “heterocyclic group”, “heterocyclic moiety”, “heterocyclic”, or “heterocyclo” used alone or as a suffix or prefix, refers to a radical derived from a heterocycle by removing one or more hydrogens therefrom.

The term “heterocyclyl” used alone or as a suffix or prefix, refers a monovalent radical derived from a heterocycle by removing one hydrogen therefrom.

The term “heteroaryl” used alone or as a suffix or prefix, refers to a heterocyclyl having aromatic character.

Heterocycle includes, for example, monocyclic heterocycles such as: aziridine, oxirane, thiirane, azetidine, oxetane, thietane, pyrrolidine, pyrroline, imidazolidine, pyrazolidine, pyrazoline, dioxolane, sulfolane 2,3-dihydrofuran, 2,5-dihydrofuran tetrahydrofuran, thiophane, piperidine, 1,2,3,6-tetrahydro-pyridine, piperazine, morpholine, thiomorpholine, pyran, thiopyran, 2,3-dihydropyran, tetrahydropyran, 1,4-dihydropyridine, 1,4-dioxane, 1,3-dioxane, dioxane, homopiperidine, 2,3,4,7-tetrahydro-1H-azepine homopiperazine, 1,3-dioxepane, 4,7-dihydro-1,3-dioxepin, and hexamethylene oxide.

In addition, heterocycle includes aromatic heterocycles (heteroaryl groups), for example, pyridine, pyrazine, pyrimidine, pyridazine, thiophene, furan, furazan, pyrrole, imidazole, thiazole, oxazole, pyrazole, isothiazole, isoxazole, 1,2,3-triazole, tetrazole, 1,2,3-thiadiazole, 1,2,3-oxadiazole, 1,2,4-triazole, 1,2,4-thiadiazole, 1,2,4-oxadiazole, 1,3,4-triazole, 1,3,4-thiadiazole, and 1,3,4-oxadiazole.

Additionally, heterocycle encompass polycyclic heterocycles, for example, indole, indoline, isoindoline, quinoline, tetrahydroquinoline, isoquinoline, tetrahydroisoquinoline, 1,4-benzodioxan, coumarin, dihydrocoumarin, benzofuran, 2,3-dihydrobenzofuran, isobenzofuran, chromene, chroman, isochroman, xanthene, phenoxathiin, thianthrene, indolizine, isoindole, indazole, purine, phthalazine, naphthyridine, quinoxaline, quinazoline, cinnoline, pteridine, phenanthridine, perimidine, phenanthroline, phenazine, phenothiazine, phenoxazine, 1,2-benzisoxazole, benzothiophene, benzoxazole, benzthiazole, benzimidazole, benztriazole, thioxanthine, carbazole, carboline, acridine, pyrolizidine, and quinolizidine.

In addition to the polycyclic heterocycles described above, heterocycle includes polycyclic heterocycles wherein the ring fusion between two or more rings includes more than one bond common to both rings and more than two atoms common to both rings. Examples of such bridged heterocycles include quinuclidine, diazabicyclo[2.2.1]heptane and 7-oxabicyclo[2.2.1]heptane.

Heterocyclyl includes, for example, monocyclic heterocyclyls, such as: aziridinyl, oxiranyl, thiiranyl, azetidinyl, oxetanyl, thietanyl, pyrrolidinyl, pyrrolinyl, imidazolidinyl, pyrazolidinyl, pyrazolinyl, dioxolanyl, sulfolanyl, 2,3-dihydrofuranyl, 2,5-dihydrofuranyl, tetrahydrofuranyl, thiophanyl, piperidinyl, 1,2,3,6-tetrahydro-pyridinyl, piperazinyl, morpholinyl, thiomorpholinyl, pyranyl, thiopyranyl, 2,3-dihydropyranyl, tetrahydropyranyl, 1,4-dihydropyridinyl, 1,4-dioxanyl, 1,3-dioxanyl, dioxanyl, homopiperidinyl, 2,3,4,7-tetrahydro-1H-azepinyl, homopiperazinyl, 1,3-dioxepanyl, 4,7-dihydro-1,3-dioxepinyl, and hexamethylene oxidyl.

In addition, heterocyclyl includes aromatic heterocyclyls or heteroaryl, for example, pyridinyl, pyrazinyl, pyrimidinyl, pyridazinyl, thienyl, furyl, furazanyl, pyrrolyl, imidazolyl, thiazolyl, oxazolyl, pyrazolyl, isothiazolyl, isoxazolyl, 1,2,3-triazolyl, tetrazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4 oxadiazolyl.

Additionally, heterocyclyl encompasses polycyclic heterocyclyls (including both aromatic or non-aromatic), for example, indolyl, indolinyl, isoindolinyl, quinolinyl, tetrahydroquinolinyl, isoquinolinyl, tetrahydroisoquinolinyl, 1,4-benzodioxanyl, coumarinyl, dihydrocoumarinyl, benzofuranyl, 2,3-dihydrobenzofuranyl, isobenzofuranyl, chromenyl, chromanyl, isochromanyl, xanthenyl, phenoxathiinyl, thianthrenyl, indolizinyl, isoindolyl, indazolyl, purinyl, phthalazinyl, naphthyridinyl, quinoxalinyl, quinazolinyl, cinnolinyl, pteridinyl, phenanthridinyl, perimidinyl, phenanthrolinyl, phenazinyl, phenothiazinyl, phenoxazinyl, 1,2-benzisoxazolyl, benzothiophenyl, benzoxazolyl, benzthiazolyl, benzimidazolyl, benztriazolyl; thioxanthinyl, carbazolyl, carbolinyl, acridinyl, pyrolizidinyl, and quinolizidinyl.

In addition to the polycyclic heterocyclyls described above, heterocyclyl includes polycyclic heterocyclyls wherein the ring fusion between two or more rings includes more than one bond common to both rings and more than two atoms common to both rings. Examples of such bridged heterocycles include quinuclidinyl, diazabicyclo[2.2.1]heptyl; and 7-oxabicyclo[2.2.1]heptyl.

The term “six-membered” used as prefix refers to a group having a ring that contains six ring atoms.

The term “five-membered” used as prefix refers to a group having a ring that contains five ring atoms.

A five-membered heteroaryl ring is a heteroaryl with a ring having five ring atoms wherein 1, 2 or 3 ring atoms are independently selected from N, O and S. Exemplary five-membered ring heteroaryls are thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4-oxadiazolyl.

A six-membered ring heteroaryl is a heteroaryl with a ring having six ring atoms wherein 1, 2 or 3 ring atoms are independently selected from N, O and S. Exemplary six-membered ring heteroaryls are pyridyl, pyrazinyl, pyrimidinyl, triazinyl and pyridazinyl.

The term “aralkyl” refers to an alkyl group substituted with an aryl group.

The term “heteroaralkyl” refers to an alkyl group substituted with an heteroaryl group.

Unless otherwise specified, the term “substituted”, when used as a prefix, refers to a structure, molecule or group, wherein one or more hydrogens are replaced with one or more alkyl groups, or one or more chemical groups containing one or more heteroatoms selected from N, O, S, F, Cl, Br, I, and P. Exemplary chemical groups containing one or more heteroatoms include heterocyclyl, —NO₂, —O-alkyl, halo, —CF₃, —CO₂H, —CO₂R, —NH₂, —SH, —NHR, —NR₂, —SR, —SO₃H, —SO₂R, —S(O)R, —CN, —OH, —C(O)NR₂, —NRC(O)R, oxo (═O), imino (═NR), thio (═S), and oximino (═N—OR), wherein each “R” is alkyl as defined above. For example, substituted phenyl may refer to nitrophenyl, pyridylphenyl, methoxyphenyl, chlorophenyl, aminophenyl, an so on, wherein the nitro, pyridyl, methoxy, chloro, and amino groups may replace any suitable hydrogen on the phenyl ring.

The term “alkoxy” used alone or as a suffix or prefix, refers to radicals of the general —O-alkyl, Exemplary alkoxy groups includes methoxy, ethoxy, propoxy, isopropoxy, butoxy, t-butoxy, isobutoxy, cyclopropylnethoxy, allyloxy, and propargyloxy.

The term “amine” or “amino” used alone or as a suffix or prefix, refers —NH₂.

The term “alkylamino” used alone or as a suffix or prefix, refers —NH(alkyl). The term “dialkylamino” used alone or as a suffix or prefix, refers —NH(alkyl)₂.

“Acyl” used alone, as a prefix or suffix, means —C(O)—R, wherein R hydrogen, hydroxyl, amino, alkylamino, dialkylamino, or alkoxy, any of which may be substituted as provided by the definition of “substituted” given above. Acyl groups include, for example, acetyl, propionyl, benzoyl, phenyl acetyl, carboethoxy, and dimethylcarbamoyl.

Some of the compounds in the present invention may exist as stereoisomers, including enantiomers, diastereomers, and geometric isomers. All of these forms, including (R), (S), epimers, diastereomers, cis, trans, syn, anti, solvates (including hydrates), tautomers, and mixtures thereof, are contemplated in the compounds of the present invention.

The invention also relates to salts of the compounds of the invention and, in particular, to pharmaceutically acceptable salts. A “pharmaceutically acceptable salt” is a salt that retains the desired biological activity of the parent compound and does not impart any undesired toxicological effects. The salts can be, for example, salts with a suitable acid, such as hydrochloric acid, hydrobromic acid, sulfuric acid, phosphoric acid, nitric acid, and the like; acetic acid, oxalic acid, tartaric acid, succinic acid, malic acid, benzoic acid, pamoic acid, alginic acid, methanesulfonic acid, naphthalenesulfonic acid, and the like. Also included are salts of cations such as ammonium, sodium, potassium, lithium, zinc, copper, barium, bismuth, calcium, and the like; or organic cations such as tetralkylammonium and trialkylammonium cations. Combinations of the above salts are also useful. Salts of other acids and/or cations are also included, such as salts with trifluoroacetic acid, chloroacetic acid, and trichloroacetic acid. The invention also includes different crystal forms, hydrates, and solvates of the compounds of the invention.

### Invention Compounds

In some embodiments, the present invention provides a compound of formula I:

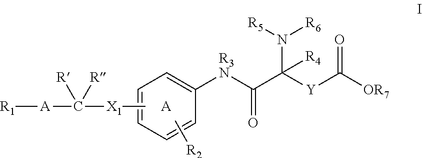

and pharmaceutically acceptable salts, phosphate derivatives, phosphate mimics, or phosphate precursor analogs thereof, wherein:

R₁ is hydrogen, halogen, cyano, alkyl, aryl, heteroaryl, cycloalkyl, heterocycloalkyl, aralkyl, heteroalkyl, —O-alkyl, —O-aryl, —O-heteroaryl, —S-alkyl, alkylene-O-alkyl, alkylene-CO₂H, alkylene-CO₂alkyl, alkylSO₂, alkylenesulfonyl, alkylene-CO-amino, alkylene-CO-alkylamino, alkylene-CO-dialkylamino, alkylene-NH—CO₂H, alkylene-NH—CO₂alkyl —CO₂alkyl, —OH, —C(O)-alkyl, —C(O)O -alkyl, —CONH₂, —CO-alkylamino, —CO-dialkylamino, amino, alkylamino, or dialkylamino, any of which may be optionally substituted on carbon with 1, 2, or 3 groups selected from halo, alkyl, OH, or —O-alkyl, which may itself be substituted on carbon with halo;

A is (C₁-C₂₀)alkylene, (C₂-C₂₀)alkenylene, or (C₂-C₂₀)alkynylene, each of which may be optionally substituted on carbon with 1, 2, or 3 groups selected from OH, CO₂H, CO₂alkyl, halogen, amino, alkylamino, dialkylamino, —O-alkyl, alkylene-O-alkyl, alkylene-OH, or alkylene-CO₂H;

X₁ is a bond or is CH₂, O, —CH₂O—, S, —S(O), —S(O)₂, —C(O)—, —C(O)O—, or NRₓ, wherein Rₓ is H or (C₁-C₆)alkyl;

R′ and R″ are each independently hydrogen, halogen, alkyl optionally substituted on carbon with halogen, alkyl, or taken together with the carbon to which they are attached form C═O or a 3, 4, 5,or 6-membered ring, optionally containing 1 or 2 heteroatoms selected from ONH, N-alkyl, SO, or SO₂, any of which may be optionally substituted on carbon with alkyl or halogen;

R₂ₐ is halogen, cyano, alkyl, aryl, heteroaryl, cycloalkyl, heterocycloalkyl, aralkyl, heteroalkyl, —O-alkyl, —O-aryl, —O-heteroaryl, aralkoxy, heteroaralkoxy, —S-alkyl, alkylene-O-alkyl, alkylene-CO₂H, alkylene-CO₂alkyl, alkylSO₂, alkylenesulfonyl, alkylene-CO-amino, alkylene-CO-alkylamino, alkylene-CO-dialkylamino, alkylene-NH—CO₂H, alkylene-NH—CO₂alkyl —CO₂alkyl, —OH, —C(O)-alkyl, —C(O)O-alkyl, —CONH₂, —CO-alkylamino, —CO-dialkylamino, amino, alkylamino, and dialkylamino, any of which may be optionally substituted on carbon with 1, 2, or 3 groups selected from halo, alkyl, OH, or —O-alkyl;

R_{2b} is hydrogen, halogen, cyano, alkyl, aryl, heteroaryl, cycloalkyl, heterocycloalkyl, aralkyl, heteroalkyl, —O-alkyl, —O-aryl, —O-heteroaryl, aralkoxy, heteroaralkoxy, —S-alkyl, alkylene-O-alkyl, alkylene-CO₂H, alkylene-CO₂alkyl, alkylSO₂, alkylenesulfonyl, alkylene-CO-amino, alkylene-CO-alkylamino, alkylene-CO-dialkylamino, alkylene-NH—CO₂H, alkylene-NH—CO₂alkyl —CO₂alkyl, —OH, —C(O)-alkyl, —C(O)O-alkyl, —CONH₂, —CO-alkylamino, —CO-dialkylamino, amino, alkylamino, and dialkylamino, any of which may be optionally substituted on carbon with 1, 2, or 3 groups selected from halo, alkyl, OH, or —O-alkyl;

R₃ is hydrogen, alkyl, or cycloalkyl, either of which may be optionally substituted on carbon with 1, 2, or 3 groups selected from halo, alkyl, OH, or —O-alkyl;

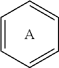

is phenyl or pyridyl;

Y is C₁-C₄ alkylene, optionally substituted on carbon with 1, 2, or 3 groups selected from halo, alkyl, OH, or —O-alkyl;

R₄ is hydrogen;

R₅ and R₆ are each independently selected from the group consisting of hydrogen, alkyl, alkylene-OH, aryl, alkylene-O-alkyl, —CO₂H, CO₂-alkyl, alkylene-OC(O)alkyl, cycloalkyl, heterocyclo, —C(O)-alkyl, —C(O)-aryl, C(O)-aralkyl, —C(O)—Oalkyl, —C(O)—Oaryl, —C(O)—Oaralkyl, alkylene-amino, alkylene-alkylamino, and alkylene-dialkylamino, any of which may be optionally substituted on carbon with halogen, alkyl, hydroxyl, CO₂H, CO₂alkyl or alkoxy; or

R₅ and R₆, together with the nitrogen to which they are attached, may form a 3, 4, 5, or 6-membered saturated or unsaturated ring, optionally containing 1 or 2 additional heteroatoms selected from O, S, NH, or N-alkyl, and optionally substituted on carbon with halogen, alkyl, hydroxyl, or alkoxy; R₅ and R₃ may form a 5, 6, 7, or 8-membered saturated or unsaturated ring, optionally containing 1 or 2 additional heteroatoms selected from O, S, NH, or N-alkyl, and optionally substituted on carbon with halogen, alkyl, hydroxyl, or alkoxy; and

R₇ is selected from the group consisting of hydrogen, alkyl, aryl or aralkyl.

In some embodiments, R₁ is hydrogen. In other embodiments, R₁ is phenyl. In still other embodiments, R₁ is pyridyl. In further embodiments, R₁ is thiophenyl. In other embodiments, R₁ is cyclohexyl. In yet other embodiments, R₁ is cyclopentyl. In some embodiments, A is n-octyl. In other embodiments, A is n-heptyl. In yet other embodiments, A is n-hexyl. In still other embodiments, A is n-pentyl. In other embodiments, A is n-butyl.

In some embodiments, X₁ is O. In other embodiments, X₁ is CH₂. In still other embodiments, X₁ is C═O. In other embodiments, X₁ is CH₂O, wherein either the oxygen or the carbon may be attached to

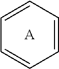

In some embodiments, R′ is hydrogen. In other embodiments, R′ is methyl. In some embodiments, R″ is hydrogen. In other embodiments, R″ is methyl.

In some embodiments, R′ and R″ taken together with the carbon to which they are attached, is C═O, with the provision that only one of X₁ or R′ and R″ taken together with the carbon may form C═O.

The compounds of the present invention include R₂ₐ which is a selectivity enhancing moiety. The term “selectivity enhancing moiety (SEM)” is defined in U.S. application Ser. No. 11/349,069 filed on Feb. 6, 2006 (published as U.S. Publication No. 20060223866) which is assigned to the assignee of the present application, the entire contents of which are incorporated herein by reference. This term refers to one or more moieties that provide an enhancement in the selectivity of the compound to which they are attached for the S1P1 receptor, as compared to the compound not containing the moiety or moieties. The SEM confers selectivity to the compound to which it is attached for the S1P1 receptor as compared to, for example, the S1P2 to S1P5 receptors. The enhancement conferred to a compound by the SEM may be measured by, for example, determining the binding specificity of a compound for the S1P1 receptor and one or more of the other S1P receptors wherein enhancement conferred to a compound by the SEM may be in the form of increased potency. The SEM of the present application is defined in one embodiment as for R.

More specifically, in some embodiments, the SEM is a halo-substituted alkyl group such as CF₃, CF₂CF₃, CF₂CF₂CF₃, CFHCF₃, CH₂CF₃, CH₂CH₂CF₃, CHCl₂, or CH₂Cl.

In certain embodiments, the SEM may possess a selectivity enhancing orientation (SEO). The term “selectivity enhancing orientation” or “SEO,” is defined in U.S. application Ser. No. 11/349,069 filed on Feb. 6, 2006 (published as U.S. Publication No. 20060223866) which is assigned to the assignee of the present application, the entire contents of which are incorporated herein by reference. This term, as used herein, refers to the relative selectivity enhancement of a compound based on the orientation of the SEM as well as the additional substituents on the ring, either alone or in combination with each other. In particular, the SEO may result from the orientation of the SEM on the ring to which it is attached, in relation to any other ring and/or moiety attached to the same ring. In one embodiment, the SEM on

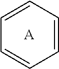

is in the ortho position relative to X₁ in Formula I. In another specific embodiment, the SEM is in the meta position relative to X₁.

Thus, in some embodiments, R₂ₐ is trifluoromethyl. In other embodiments, R₂ is fluoro. In still other embodiments, R₂ is chloro. In further embodiments, R₂ is bromo. In other embodiments, R₂ is cyano. In yet other embodiments, R₂ is methyl.

In some embodiments, R₃ is hydrogen. In other embodiments, R₃ is alkyl.

In some embodiments, R₄ is hydrogen.

In some embodiments, R₅ and R₆ are each independently hydrogen.

In some embodiments, R₇ is H. In other embodiments, R₇ is methyl. In still other embodiments, R₇ is ethyl. In other embodiments, R₇ is benzyl.

In some embodiments,

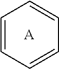

is phenyl. In other embodiments,

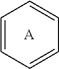

is pyridyl.

In some embodiments, Y is CH₂.

In some embodiments, compounds of the invention are compounds wherein

R₁ is hydrogen, aryl, cycloalkyl, or heteroaryl.

R₄ is hydrogen;

R₅ and R₆ are each independently hydrogen or alkyl, or alkylene-OH;

In other embodiments, compounds of the invention are compounds wherein

R₁ is hydrogen or aryl;

R₄ is hydrogen;

R₅ and R₆ are each independently hydrogen or alkyl, or alkylene-OH;

R₇ is hydrogen.

In further embodiments, compounds of the invention are compounds wherein

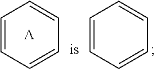

R₁ is phenyl;

A is (C₁-C₁₀)alkyl;

R′ and R″ are hydrogen;

X₁ is CH₂ or O;

R₃ is hydrogen or alkyl;

Y is CH₂;

R₄ is hydrogen;

R₅ and R₆ are each independently hydrogen, alkyl;

In some embodiments, the compounds of the present invention include compounds in the following table:

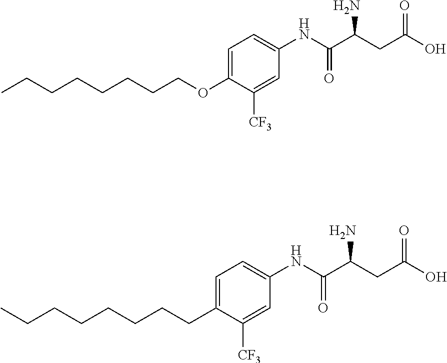

as well as pharmaceutically acceptable salts, phosphate derivatives, phosphate mimics, or phosphate precursor analogs thereof.

### Biological Activity of Invention Compounds

Lymphopenia Assay

Several of the compounds described herein were evaluated for the ability to induce lymphopenia in mice. Male C57B1/6 mice were divided into groups of three. A control group received the 3% BSA vehicle only. The other groups received a single dose of either a specified dose of test compound in vehicle administered orally (PO) and intravenously (IV). After 6 hours, the mice were anesthesized with isoflurane and approximately 250 μL of blood was removed from the retroorbital sinus and collected in an EDTA microtainer, mixed with an anticoagulant and placed on a tilt table until complete blood count (CBC) analysis. Oral administration (10 mg/K) of these compounds induced increased lymphopenia versus the vehicle.

Binding to S1P1 or S1P3 Receptors

In certain embodiments, the compounds of the invention selective for the S1P1 receptor as compared to one or more of the other S1P receptors. For example, one set of compounds includes compounds which are selective for the S1P1 receptor relative to the S1P3 receptor. Compounds selective for the S1P1 receptor can be agonists of the S1P1 receptor, significantly weaker agonists of one or more other receptors and/or antagonists of one or more other receptors. A compound is “selective” for the S1P1 receptor relative to a second receptor, if the EC₅₀ of the compound for the second receptor is at least two-fold greater than the EC₅₀ for the S1P1 receptor. The EC₅₀ of a compound is determined using the ³⁵S-GTPγS binding assay, as described in WO 03/061567, the entire contents of which are incorporated herein by reference. Additionally or alternatively, a compound is “selective” for the S1P1 receptor relative to a second receptor, if the IC₅₀ of the compound for the second receptor is at least two-fold greater than the IC₅₀ for the S1P1 receptor. The IC₅₀ of a compound is determined using the [³³P]sphingosine 1-phosphate binding assay, as described in Davis, M. D. et al., Sphingosine 1-Phosphate Analogs as Receptor Antagonists. J. Biol. Chem. (2005) 280:9833-9841, the entire contents of which are incorporated herein by this reference.

The terms “agonist” or “S1P1 receptor agonist” as used herein include the compounds described herein which bind to and/or agonize the S1P1 receptor. In one embodiment, the SIP receptor agonists have an IC₅₀ for the S1P1 receptor of about 100 nM-0.25 nM, about 50 nM-0.25 nM, about 25 nM-0.5 nM, about 100 nM or less, about 75 nM or less, about 50 nM or less, about 40 nM or less, about 30 nM or less, about 20 nM or less, about 10 nM or less, about 5 nM or less, about 1 nM or less, about 0.5 nM or less, or about 0.25 nM or leis. The compounds' IC₅₀ for the S1P1 receptor can be measured using the binding assays described in Example 13 or those described in WO 03/061567. Compounds of the invention generally had an IC₅₀ in the range of 100 pM (picomolar) to 100 M.

For example,

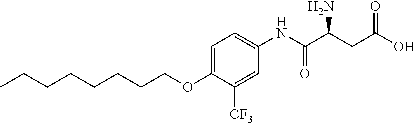

had an IC₅₀ of value of 6:6 nM

Ranges intermediate to the above recited values are also intended to be part of this invention. For example, ranges using a combination of any of the above recited values as upper and/or lower limits are intended to be included.

In a further embodiment, the SIP receptor agonist has an IC₅₀ value for the S1P3 receptor of about 10 nM-10,000 nM, about 100 nM-5000 nM, about 100 nM-3000 nM, about 10 nM or greater, about 20 nM or greater, about 40 nM or greater, about 50 nM or greater, about 75 nM or greater, or about 100 nM or greater. In another embodiment, the S1P compound of the invention binds the S1P3 receptor with an IC₅₀ of 1000 nM or greater, 2000 nM or greater, 3000 nM or greater, 5000 nM or greater, 10,000 nM or greater. The IC₅₀ for of S1P3 receptor can be measured using the binding assays described herein or those described in WO 03/061567.

In addition, it should be understood that the ranges intermediate to the above recited values are also intended to be part of this invention. For example, ranges using a combination of any of the above recited values as upper and/or lower limits are intended to be included.

In yet another embodiment, the S1P receptor agonists described herein have an IC₅₀ value for the S1P1 receptor that is about 5-fold lower, about 10-fold lower, about 20-fold lower, about 50-fold lower, about 100-fold lower, about 200-fold lower, about 500-fold lower or about 1000-fold lower than their IC₅₀ value for the S1P3 receptor.

Ranges intermediate to the above recited values are also intended to be part of this invention. For example, ranges using a combination of any of the above recited values as upper and/or lower limits are intended to be included.

The ability of several of the compounds described herein to bind to the S1P1 or S1P3 receptor was also tested as follows.

For the membrane preparation, plasmid DNA was transfected into HEK 293 T cells using the FuGENE 6 transfection protocol (publicly available from Roche). Briefly, subconfluent monolayers of HEK 293 T cells were transfected with the DNA mixture containing FuGENE 6 (using a 1:3 ratio). The dishes containing the cells were then placed in a tissue culture incubator (5% CO₂, 37° C.). The cells were harvested 48 hours after addition of the DNA by scraping in HME buffer (in mM: 20 HEPES, 5 MgCl₂, 1 EDTA, pH 7.4, 1 mM PMSF) containing 10% sucrose on ice, and disrupted using a Dounce homogenizer. After centrifugation at 800×g, the supernatant was diluted with HME without sucrose and centrifuged at 17,000×g for 1 hour. This crude membrane pellet was resuspended in HME with sucrose, aliquoted, and snap-frozen by immersion in liquid nitrogen. The membranes were stored at −70 C. Protein concentration was determined spectroscopically by Bradford protein assay.

For the binding assay, [³³P]sphingosine 1-phosphate (obtained from American Radiolabeled Chemicals, Inc) was added to membranes in 200 μl in 96-well plates with assay concentrations of 2.5 pM [³³P]sphingosine 1-phosphate, 4 mg/ml BSA, 50 mM HEPES, pH 7.5, 100 mM NaCl, 5 mM MgCl2, and 5 μg of protein. Binding was performed for 60 minutes at room temperature with gentle mixing and terminated by collecting the membranes onto GF/B filter plates. After drying the filter plates for 10 minutes, 50 μl of Microscint 40 was added to each well, and filter-bound radionuclide was measured on a Packard Top Count. Nonspecific binding was defined as the amount of radioactivity remaining in the presence of excess of unlabeled S1P.

In one embodiment, the compounds of the invention do not include the compounds described in WO 06/020951A2, WO 05/041899A2, WO 04/010949A2, WO 04/024673 A1 and WO 02/064616, and U.S. Ser. No. 11/349,069, filed Feb. 2, 2006; the entire contents of each of which are hereby incorporated herein by reference.

### Methods of Using Invention Compounds

The compounds of the invention have been determined to be useful in the treatment of sphingosine 1-phosphate associated disorders. Accordingly, in one embodiment, the invention relates to a method for treating a subject suffering from a sphingosine 1-phosphate associated disorder, comprising administering to a subject an effective amount of a compound of the invention; that is, a compound of formula I or compounds otherwise described herein, such that the subject is treated for a sphingosine I-phosphate associated disorder.

The term “sphingosine 1-phosphate associated disorder” includes disorders, diseases or conditions which are associated with or caused by a misregulation in S1P receptor function and/or signaling or S1P receptor ligand function. The term also includes diseases, disorders or conditions which can be treated by administering to a subject an effective amount of a sphingosine 1-phosphate receptor agonist. Such disorders include disorders that are associated with an inappropriate immune response and conditions associated with an overactive immune response, e.g., autoimmune diseases.

“Treatment”, or “treating” as used herein, is defined as the application or administration of a therapeutic agent such as a compound of formula I to a subject who has a shingosine 1-phosphate associated disorder as described herein, with the purpose to cure, heal, alleviate, delay, relieve, alter, remedy, ameliorate, improve or affect the disease or disorder, or symptoms of the disease or disorder. The term “treatment” or “treating” is also used herein in the context of administering agents prophylactically.

In some embodiments, the efficacy of the compounds of the present invention can be measured by comparing a value, level, feature, characteristic, property, etc. to a “suitable control”. A “suitable control” is any control or standard familiar to one of ordinary skill in the art useful for comparison purposes. In one embodiment, a “suitable control” is a value, level, feature, characteristic, property, etc. determined prior to administering a composition of the present invention. For example, the immune response, etc. can be determined prior to introducing a compound of the invention into a cell or subject. In another embodiment, a “suitable control” is a value, level, feature, characteristic, property, etc. determined in a cell or organism, e.g., a control or normal cell or organism, exhibiting, for example, normal traits. In yet another embodiment, a “suitable control” is a predefined value, level, feature, characteristic, property, etc. For example a “suitable control” can be a pre-defined level of binding to a specified S1P receptor.

An additional embodiment of the invention pertains to a method for treating a subject suffering from a sphingosine 1-phosphate associated disorder, comprising administering to a subject a compound, such that the subject is treated for a sphingosine 1-phosphate associated disorder by a compound of the invention; that is, a compound of formulae I or compounds otherwise described herein.

The present invention is also directed to a method of selectively treating a sphingosine 1-phosphate associated disorder, comprising administering to a subject an effective amount of a compound of the invention, e.g., compounds of any of Formulae I-VIII or compounds otherwise described herein, such that the subject is selectively treated for a sphingosine 1-phosphate associated disorder. In certain embodiments, the sphingosine 1-phosphate associated disorder is a sphingosine 1-phosphate-(1) associated disorder. In a particular embodiment, the sphingosine 1-phosphate-(1) associated disorder is selectively treated as compared with a sphingosine 1-phosphate-(3) associated disorder.

Another embodiment of the invention is a method of selectively treating a sphingosine 1-phosphate associated disorder, comprising administering to a subject a compound, such that the subject is selectively treated for a sphingosine 1-phosphate associated disorder by a compound of the invention, e.g., compounds of any of Formulae I-VIII or compounds otherwise described herein. In certain embodiments, the sphingosine 1-phosphate associated disorder is a sphingosine 1-phosphate-(1) associated disorder. In a particular embodiment, the sphingosine 1-phosphate-(1) associated disorder is selectively treated as compared with a sphingosine 1-phosphate-(3) associated disorder.

In another embodiment, the present invention provides a method of treating a condition associated with an activated immune system. Such diseases or disorders include rejection of transplanted organs, tissue or cells; graft-versus-host diseases brought about by transplantation; autoimmune syndromes including rheumatoid arthritis; systemic lupus erythematosus; antiphospholipid syndrome; Hashimoto's thyroiditis; Iymphocytic thyroiditis; multiple sclerosis; myasthenia gravis; type I diabetes; uveitis; episcleritis; scleritis; Kawasaki's disease, uveo-retinitis; posterior uveitis; uveitis associated with Behcet's disease; uveomeningitis syndrome; allergic encephalomyelitis; chronic allograft vasculopathy; post-infectious autoimmune diseases including rheumatic fever and post-infectious glomerulonephritis; inflammatory and hyperproliferative skin diseases; psoriasis; psoriatic arthritis; atopic dermatitis; myopathy; myositis; osteomyelitis; contact dermatitis; eczematous dermatitis; seborrhoeic dermatitis; lichen plan us; pemphigus; bullous pemphigoid; epidermolysis bullosa; urticaria; angioedema; vasculitis; erythema; cutaneous eosinophilia; acne; scleroderma; alopecia areata; keratoconjunctivitis; vernal conjunctivitis; keratitis; herpetic keratitis; dystrophia epithelialis corneas; corneal leukoma; ocular pemphigus; Mooren's ulcer; ulcerative keratitis; scleritis; Graves' ophthalmopathy; Vogt-Koyanagi-Harada syndrome; sarcoidosis; pollen allergies; reversible obstructive airway disease; bronchial asthma; allergic asthma; intrinsic asthma; extrinsic asthma; dust asthma; chronic or inveterate asthma; late asthma and airway hyper-responsiveness; bronchiolitis; bronchitis; endometriosis; orchitis; gastric ulcers; ischemic bowel diseases; inflammatory bowel diseases; necrotizing enterocolitis; intestinal lesions associated with thermal burns; coeliac disease; proctitis; eosinophilic gastroenteritis; mastocytosis; Crohn's disease; ulcerative colitis; vascular damage caused by ischemic diseases and thrombosis; atherosclerosis; fatty heart; myocarditis; cardiac infarction; aortitis syndrome; cachexia due to viral disease; vascular thrombosis;. migraine; rhinitis; eczema; interstitial nephritis; IgA-induced nephropathy; Goodpasture's syndrome; hemolytic-uremic syndrome; diabetic nephropathy; glomerulosclerosis; glomerulonephritis; tubulointerstitial nephritis; interstitial cystitis; multiple myositis; Guillain-Barre syndrome; Meniere's disease; polyneuritis; multiple neuritis; myelitis; mononeuritis; radiculopathy; hyperthyroidism; Basedow's disease; thyrotoxicosis; pure red cell aplasia; aplastic anemia; hypoplastic anemia; idiopathic thrombocytopenic purpura; autoimmune hemolytic anemia; autoimmune thrombocytopenia; agranulocytosis; pernicious anemia; megaloblastic anemia; anerythroplasia; osteoporosis; fibroid lung; idiopathic interstitial pneumonia; dermatomyositis; leukoderma vulgaris; ichthyosis vulgaris; photoallergic sensitivity; cutaneous T cell lymphoma; polyarteritis nodosa; Huntington's chorea; Sydenham's chorea; myocardosis; myocarditis; scleroderma; Wegener's granuloma; Sjogren's syndrome; adiposis; eosinophilic fascitis; lesions of gingiva, periodontium, alveolar bone, substantia ossea dentis; male pattern alopecia or alopecia senilis; muscular dystrophy; pyoderma; Sezary's syndrome; hypophysitis; chronic adrenal insufficiency; Addison's disease; ischemia-reperfusion injury of organs which occurs upon preservation; endotoxin shock; pseudomembranous colitis; colitis caused by drug or radiation; ischemic acute renal insufficiency; chronic renal insufficiency; lung solid cancer; malignancy of lymphoid origin; acute or chronic Iymphocytic leukemias; lymphoma; psoriasis; pulmonary emphysema; cataracts; siderosis; retinitis pigmentosa; senile macular degeneration; vitreal scarring; corneal alkali burn; dermatitis erythema; ballous dermatitis; cement dermatitis; gingivitis; periodontitis; sepsis; pancreatitis; peripheral artery disease; carcinogenesis; solid cancer tumors; metastasis of carcinoma; hypobaropathy; autoimmune hepatitis; primary biliary cirrhosis; sclerosing cholangitis; partial liver resection; acute liver necrosis; cirrhosis; alcoholic cirrhosis; hepatic failure; fulminant hepatic failure; late-onset hepatic failure; “acute-on-chronic” liver failure.

As used herein, the term “subject” includes warm-blooded animals, e.g., mammals, including humans, cats, dogs, horses, bears, lions, tigers, ferrets, rabbits, mice, cows, sheep, pigs, etc. In a particular embodiment, the subject is a primate. In a specific embodiment, the primate is a human.

As used herein, the term “administering” to a subject includes dispensing, delivering or applying a compound of the invention in a pharmaceutical formulation (as described herein), to a subject by any suitable route for delivery of the compound to the desired location in “the subject, including delivery by either the parenteral or oral route, intramuscular injection, subcutaneous/intradermal injection, intravenous injection, buccal administration, topical delivery, transdermal delivery and administration by the rectal, colonic, vaginal, intranasal or respiratory tract route.

As used herein, the term “effective amount” includes an amount effective, at dosages and for periods of time necessary, to achieve the desired result, e.g., sufficient to treat the condition in a subject. An effective amount of a compound of the invention, as defined herein, may vary according to factors such as the disease state, age, and weight of the subject, and the ability of the compound to elicit a desired response in the subject. Dosage regimens may be adjusted to provide the optimum therapeutic response. An effective amount is also one in which any toxic or detrimental effects (e.g., side effects) of the compound are outweighed b_{y} the therapeutically beneficial effects.

A therapeutically effective amount of a compound of the invention (i.e., an effective dosage) may range from about 0.001 to 30 mg/kg body weight, for example, about 0.01 to 25 mg/kg body weight, for example, about 0.1 to 20 mg/kg body weight. It is to be understood that all values and ranges between those listed are intended to be encompassed by the present invention. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a compound of the invention can include a single treatment or, for example, can include a series of treatments. It will also be appreciated that the effective dosage of the compound used for treatment may increase or decrease over the course of a particular treatment.

The methods of the invention further include administering to a subject a therapeutically effective amount of a compound of the invention in combination with another pharmaceutically active compound known to treat the disease or condition, e.g., an immunomodulatory agent or an anti-inflammatory agent. Pharmaceutically active compounds that may be used depend upon the condition to be treated, but include as examples cyclosporin, rapamycin, FK506, methotrexate, etanercept, infliximab, adalimumab, non-steroidal anti-inflammatory agents, cyclooxygenase-2-inhibitors, such as celecoxib and rofecoxib, and corticosteroids. Other suitable compounds can be found in Harrison's Principles of Internal Medicine, Thirteenth Edition, Eds. T. R. Harrison et al. McGraw-Hill N.Y., N.Y.; and the Physicians Desk Reference 50th Edition 1997, Oradell N.J., Medical Economics Co., the complete contents of which are expressly incorporated herein by reference. The compound of the invention and the additional pharmaceutically active compound may be administered to the subject in the same pharmaceutical composition or in different pharmaceutical compositions (at the same time or at different times).

### Pharmaceutical Compositions Comprising Invention Compounds

The present invention also provides pharmaceutically acceptable formulations and compositions comprising one or more compounds of the invention, for example, compounds of formula I or compounds otherwise described herein. In certain embodiments, the compound of the invention is present in the formulation in a therapeutically effective amount; that is, an amount effective to treat a sphingosine 1-phosphate associated disorder.

Accordingly, in one embodiment, the invention pertains to a pharmaceutical composition comprising a therapeutically effective amount of a compound of the invention; that is, compounds of formula I or compounds otherwise described herein, and a pharmaceutically acceptable carrier.

In another embodiment, the invention is directed to a packaged pharmaceutical composition comprising a container holding a therapeutically effective amount of a compound of the invention; that is, compounds of formula I or compounds otherwise described herein; and instructions for using the compound to treat a sphingosine 1-phosphate associated disorder in a subject.

The term “container” includes any receptacle for holding the pharmaceutical composition. For example, in one embodiment, the container is the packaging that contains the pharmaceutical composition. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. Moreover, packaging techniques are well known in the art. It should be understood that the instructions for use of the pharmaceutical composition may be contained on the packaging containing the pharmaceutical composition, and as such the instructions form an increased functional relationship to the packaged product. However, it should be understood that the instructions can contain information pertaining to the compound's ability to perform its intended function, e.g., treating, preventing, or reducing a sphingosine 1-phosphate associated disorder in a subject.

Another embodiment of the invention relates to a packaged pharmaceutical composition comprising a container holding a therapeutically effective amount of a compound of the invention; that is, a compound of formula I or compounds otherwise described herein, and instructions for using the compound to selectively treat a sphingosine 1-phosphate associated disorder in a subject.

Such pharmaceutically acceptable formulations typically include one or more compounds of the invention as well as one or more pharmaceutically acceptable carriers and/or excipients. As used herein, “pharmaceutically acceptable carrier” includes any and all solvents, dispersion media, coatings, antibacterial and antifungal agents, isotonic and absorption delaying agents, and the like that are physiologically compatible. The use of such media and agents for pharmaceutically active substances is well known in the art. Except insofar as any conventional media or agent is incompatible with the compounds of the invention, use thereof in the pharmaceutical compositions is contemplated.

Supplementary pharmaceutically active compounds known to treat transplant or autoimmune disease, i.e., immunomodulatory agents and anti-inflammatory agents, as described above, can also be incorporated into the compositions of the invention. Suitable pharmaceutically active compounds that may be used can be found in Harrison's Principles of Internal Medicine.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injection include sterile aqueous solutions (where water soluble) or dispersions, or sterile powders for the extemporaneous preparation of sterile injectable solutions or dispersions. For intravenous administration, suitable carriers include physiological saline, bacteriostatic water, Cremophor E1™ (BASF, Parsippany, N.J.) or phosphate buffered saline (PBS). In all cases, the pharmaceutical composition must be sterile and should be fluid to the extent that easy syringability exists. It must also be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, or sodium chloride in the composition. Prolonged absorption of the injectable compositions can be brought about by including in the composition an agent which delays absorption, for example, aluminum monostearate and gelatin.

Sterile injectable solutions can be prepared by incorporating the compound of the invention in the required amount in an appropriate solvent with one or a combination of the ingredients enumerated above, as needed, followed by filtered sterilization. Generally, dispersions are prepared by incorporating the compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the compound plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the compound of the invention can be incorporated with excipients and used in the form of tablets, troches, or capsules. Oral compositions can also include an enteric coating. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or sterates; a glidant such as colloidal silicon dioxide; a sweetening agent such as sucrose or saccharin; or a flavoring agent such as peppermint, methyl salicylate, or orange flavoring.

For administration by inhalation, the compounds of the invention are delivered in the form of an aerosol spray from a pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the compounds of the invention are formulated into ointments, salves, gels, or creams as generally known in the art.

The present pharmaceutical compositions can also be prepared in the form of suppositories (e.g., with conventional suppository bases such as cocoa butter and other glycerides) or retention enemas for rectal delivery.

In one embodiment, the compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions can also be used as pharmaceutically acceptable carriers. These can be prepared according to methods known to those skilled in the art, for example, as described in U.S. Pat. No. 4,522,811, U.S. Pat. No. 5,455,044 and U.S. Pat. No. 5,576,018, and U.S. Pat. No. 4,883,666, the entire contents of all of which are incorporated herein by reference.

The compounds of the invention can also be incorporated into pharmaceutical compositions which allow for the sustained delivery of the compounds to a subject for a period of at least several weeks to a month or more. Such formulations are described in published PCT application no. WO 02/74247, the entire contents of which are incorporated herein by reference.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of a compound of the invention calculated to produce the desired therapeutic effect in association with the required pharmaceutical carrier. The specification for the unit dosage forms of the invention are dictated by and directly dependent on the unique characteristics of the compound and the particular therapeutic effect to be achieved, and the limitations inherent in the art of compounding such compounds for the treatment of individuals.

This invention is further illustrated by the following examples, which should not be construed as limiting. The contents of all references, patents, patent applications cited throughout this application are incorporated herein by reference. It should be understood that the use of any of the compounds described herein are within the scope of the present invention and are intended to be encompassed by the present invention and are expressly incorporated herein for all purposes.

### Examples

### General Synthetic Strategy for Synthesis of Amide-Carboxylates

The general approach for synthesis of various amide-carboxylateas 6 is described in Scheme 1. Reaction of desired alcohol with substituted 1-fluoro-4-nitrobenzene 1 afforded the nitrobenzene intermediate which upon hydrogenation afforded aniline 2. Des-oxygen analogs of aniline 2 were synthesized from substituted 1-bromo- or 1-iodo-4-nitrobenzene using Sonogashira cross-coupling conditions followed by hydrogenation. Coupling of the desired aniline with the protected amino acid afforded amide 5 which, upon deprotection, gave amide 6 in good yield.

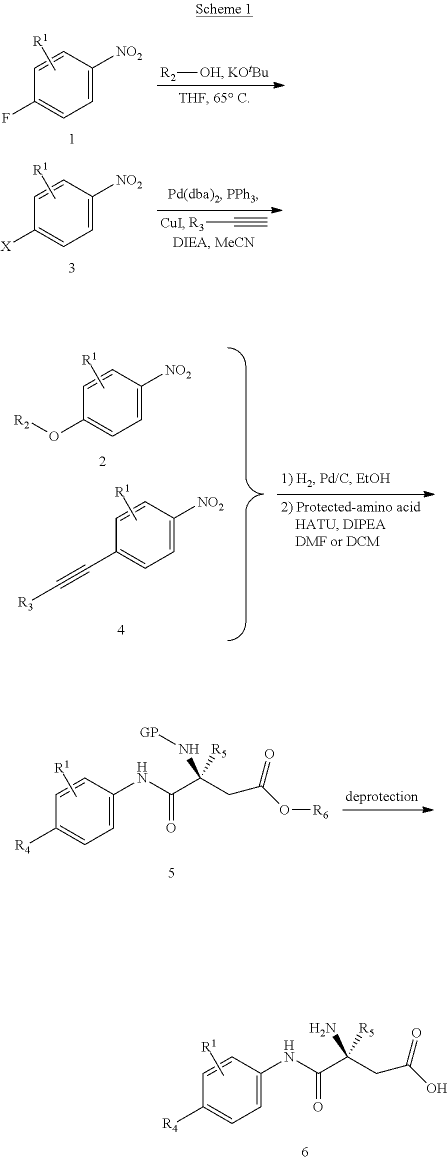

### General Protocol for Synthesis of Substituted Nitrobenzene Ether 2

To a solution of the desired alcohol (1.0 equivalent) in dry THF under nitrogen atmosphere was added KO^{t}Bu (either 1.0 M solution in THF or solid, 1.1 equivalents). The reaction mixture was heated at 60-70° C. for 10 minutes, then substituted 1-flouro-4-nitrobenzene 1 (1.0 equivalent) was added. The reaction was then stirred for 1 to 3 hours before cooling to room temperature. The solvent removed in vacuo. The product was purified by silica gel column chromatography using the Combi-Flash system (Hex:EtOAc).

### 4-Nitro-1-(octyloxy)-2-(trifluoromethyl)benzene (2a)

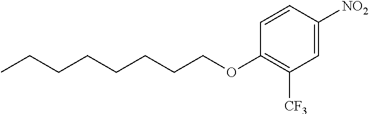

The product was purified by silica gel column chromatography using the Combi-Flash system (Hex:EtOAc) as yellow-brownish oil in 79% (1.20 g). TLC (1:4 EtOAc:Hex), R₁=0.6; ¹H NMR (400 MHz, CDCl₃) δ 7.50 (d, 1H, J=2.4 Hz), 8.40 (dd, 1H, J=9.2 Hz, J=2.4 Hz), 7.07 (d, 1H, J=9.2 Hz), 4.17 (t, 2H, J=6.4 Hz), 1.80-1.92 (m, 2H), 1.43-1.456 (m, 2H), 1.20-1.42 (m, 8H), 0.89 (t, 3H, J=6.4 Hz).

### General Protocol for Synthesis of Substituted Nitrobenzene 4

A suspension of substituted bromo- or iodo- nitrobenzene (1.0 equivalent), 1-octyne (1.5 equivalents), Pd(dba)₂ (2 mol %), Ph₃P (5 mol %), CuI (2 mol %), and diisopropylethyl amine (DIEA) (4.0 equivalents) was heated at 100° C. under microwave condition for 1.5 hours. The reaction was diluted with ethyl acetate (EtOAc) and washed with saturated NaHCO₃ aqueous solution (1 time), saturated NH₄Cl aqueous solution (1 time), and brine (1 time). The organic layer was dried over Na₂SO₄, and concentrated in vacuo. The product was purified by silica gel column chromatography using the Combi-Flash system (Hex:EtOAc).

### 4-nitro-1-(oct-1-ynyl)-2-(trifluoromethyl)benzene (4a)

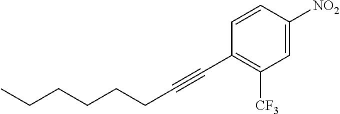

The product was purified by silica gel column chromatography using the Combi-Flash system (Hex:EtOAc) as colourless oil in 88% (1.06 g). ¹H NMR (400 MHz, CDCl₃) δ 8.49 (d, 1H, J=2.4 Hz), 8.31 (dd, 1H, J=8.4 Hz, J=2.4 Hz), 7.68 (d, 1H, J=8.8 Hz), 2.50 (t, 2H, J=6.8 Hz), 1.64 (m, 2H), 1.47 (m, 2H), 1.33 (m, 4H), 0.91 (t, 3H, J=6.8 Hz).

### General Protocol for Synthesis of Amide 5

To a solution of the nitrobenzene 2 (1.0 equivalent) in MeOH or EtOH was added 10% Pd/C (10% by weight). The reaction mixture was stirred under an atmosphere of H₂ (g) for 1-3 hours. The reaction was filtered through a thin layer of Celite then the solvent removed in vacuo. The obtained aniline was carried forward as is.

To a solution of protected amino acid (1.0 equivalent), O-(7-Azabenzotriazole-1-yl)-N,N,N′N′-tetramethyluronium hexafluorophosphate (HATU) (1.2 equivalents), and DIEA (3.0 equivalents) in dimethylformamide (DMF) or dichloromethane or a mixture of the two solvents was added the desired aniline. The resultant mixture was stirred at room temperature for 1-3 hours. The reaction was then diluted with EtOAc and washed with 10% NH₄Cl (2 times) and saturated NaCl (1 time). The solvent removed in vacuo and the product was purified by silica gel column chromatography using the Combi-Flash system (Hex:EtOAc).

### Removal of Boc- and/or t-Butyl Ester Protecting Group

To a solution of the desired starting material (1.0 equivalent) in dry dichloromethane was added trifluoroacetic acid (TFA, 30% by volume). The reaction mixture was stirred at room temperature 1-3 hours, then evaporated to dryness under reduced pressure. The obtained residue was then azeotroped with dichloromethane (2 times) to remove any excess TFA. The final product was either used as is or purified by reverse phase preparative HPLC.

### (S)-3-amino-4-(4-(octyloxy)-3-(trifluoromethyl)phenylamino)-4-oxobutanoic acid (6a)

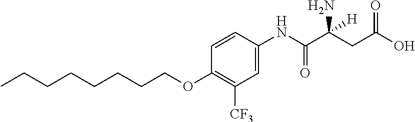

The product was obtained as white solid in 98% (0.476 g) yield over three steps. MS (ESI, M+H⁺)=405.08; ¹H NMR (400 MHz, DMSO-d₆) δ 10.56 (br s, 1H), 8.31 (br s, 3H), 7.88 (d, 1H, J=2.4 Hz), 7.71 (dd, 1H, J=8.8 Hz, J=2.4 Hz), 7.25 (d, 1H, J=8.8 Hz), 4.14-4.22 (m, 1H), 4.05 (t, 2H, J=6.4 Hz), 2.97 (dd, 1H, J=17.6 Hz, J=4.8 Hz), 2.82 (dd, 1H, J=16.0 Hz, J=7.6 Hz), 1.63-1.76 (m, 2H), 1.19-1.44 (m, 10H), 0.84 (t, 3H, J=6.4 Hz).

### (S)-3-Amino-4-(4-octyl-3-(trifluoromethyl)phenylamino)-4-oxobutanoic acid (6b)

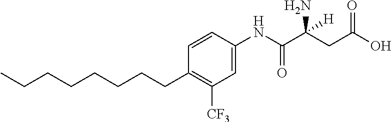

The product was obtained as white solid in 67% (45 mg) yield over three steps. MS (ESI, M+H⁺)=389.12; NMR (400 MHz, DMSO-d₆) δ 10.70 (br s, 1H), 7.96 (d, 1H, J=2.0 Hz), 7.73 (dd, 1H, J=8.4 Hz, J=2.0 Hz), 7.25 (d, 1H, J=8.6 Hz), 4.19 (dd, 1H, J=8.0 Hz, J=4.8 Hz), 2.96 (dd, 1H, J=17.2 Hz, J=4.8 Hz), 2.81 (dd, 1H, J=17.2 Hz, J=8.0 Hz), 2.68 (t, 2H, J=8.0 Hz), 1.48-1.60 (m, 2H), 1.15-1.40 (m, 10H), 0.86 (t, 3H, J=6.8 Hz).

### Synthesis of (S)-2-(benzyloxycarbonylamino)-4-tert-butoxy-2-methyl-4-oxobutanoic acid 9

The general approach for synthesis of Cbz-protected a-methyl-aspartic ester 9 is described in Scheme 2. Reaction of Cbz-protected alanine 7 with (dimethoxymethyl)benzene in presence of Lewis acid afforded oxazolidinone 8. Reaction of the oxazolidinone 8 with lithium hexamethyldisilazide (LHMDS) followed by addition of tert-butyl 2-bromoacetate and based-catalyzed opening of the oxazolidinone gave the desired amino acid 9. As a note, (R)-2-(benzyloxycarbonylamino)-4-tert-butoxy-2-methyl-4-oxobutanoic acid was prepared as provided in Altmann, E.; Nebel, K.; Mutter, M. Helvetica Chimica Acta 1991, 74, 800-806.

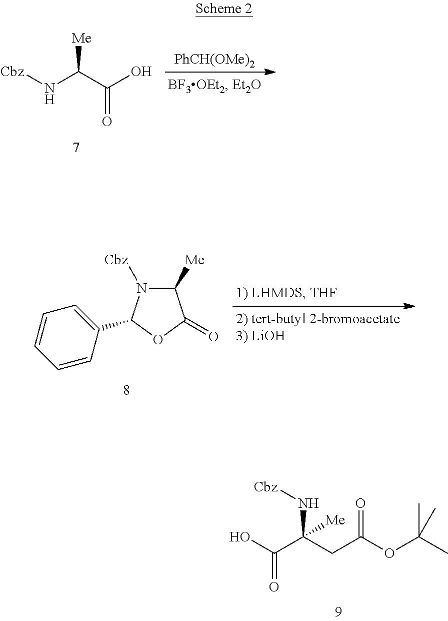

### (2R,4S)-Benzyl 4-methyl-5-oxo-2-phenyloxazolidine-3-carboxylate (8)

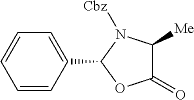

To a solution of Cbz-alanine (5.0 g) in dry diethyl ether (60 mL) was added (dimethoxymethyl)benzene (3.7 mL, 1.1 equivalents) and BF₃.OEt₂ (11.9 mL, 4.3 equivalents). The solution was stirred overnight. To the brownish solution was added Et₃N (19.0 mL, 6.0 equivalents). The reaction was then diluted with EtOAc (100 mL) and washed with 10% NH₄Cl (2×100 mL), 5% NaHCO₃ (1×100 mL), and saturated NaCl (1×100 mL). The solvent removed in vacuo and the product was purified by silica gel column chromatography using the Combi-Flash system (Hex:EtOAc). The product was obtained as yellowish oil in 94% (6.55 g) yield. TLC (1:3 EtOAc:Hex), R_{f}=0.5; ¹H NMR (400 MHz, CDCl₃) δ 7.01-7.60 (m, 10H), 6.64 (s, 1H), 5.10-5.22 (m, 2H), 4.49 (q, 1H, J=6.8 Hz), 1.58 (d, 3H, J=6.8 Hz).

### (S)-2-(Benzyloxycarbonylamino)-4-tent-butoxy-2-methyl-4-oxobutanoic acid (9)

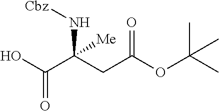

For detailed experimental procedures refer to Altmann, E.; Nebel, K.; Mutter, M. Helvetica Chimica Acta 1991, 74, 800-806. The product was obtained as colorless oil in 61% (1.05 g) yield over three steps. ¹H NMR (400 MHz, CDCl₃) δ 7.34 (s, 5H), 6.09 (br s, 1H), 5.05-5.15 (m, 2H), 3.15 (d, 1H, J=15.6 Hz), 2.94 (d, 1H, J=15.6 Hz), 1.67 (s, 3H), 1.40 (s, 9H).

### (S)-3-amino-3-methyl-4-(4-(octyloxy)-3-(trifluoromethyl)phenylamino)-4-oxobutanoic acid (6c)

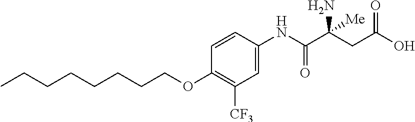

After the coupling of the aniline to the desired orthogonally protected α-methyl-aspartic ester, the Cbz-protecting group was removed first by hydrogenation followed by cleavage of the t-butyl ester with TFA. The product was obtained as white solid in 42% (0.210 g) yield over the three steps. MS (ESI, M+H⁺)=419.08; ¹H NMR (400 MHz, DMSO-d₆) δ 10.02 (br s, 1H), 8.26 (br s, 3H), 7.81. (d, 1H, J=2.4 Hz), 7.73 (dd, 1H, J=8.8 Hz, J=2.4 Hz), 7.25 (d, 1H, J=8.8 Hz), 4.06 (t, 2H, J=6.4 Hz), 3.39 (d, 1H, J=16.0 Hz), 2.93 (d, 1H, J=16.0 Hz), 1.60-1.76 (m, 2H), 1.54 (s, 3H), 1.19-1.46 (m, 10H), 0.84 (t, 3H, J=6.4 Hz).

1. A compound of formula I: 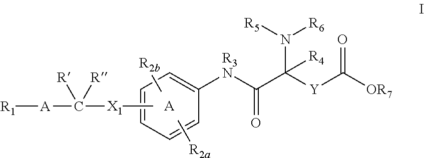
or a pharmaceutically acceptable salt, phosphate derivative, phosphate mimic, or phosphate precursor analog thereof, wherein:
R₁ is hydrogen, halogen, cyano, alkyl, aryl, heteroaryl, cycloalkyl, heterocycloalkyl, aralkyl, heteroalkyl, —O-alkyl, —O-aryl, —O-heteroaryl, —S-alkyl, alkylene-O-alkyl, alkylene-CO₂H, alkylene-CO₂alkyl, alkylSO₂, alkylenesulfonyl, alkylene-CO-amino, alkylene-CO-alkylamino, alkylene-CO-dialkylamino, alkylene-NH—CO₂H, alkylene NH—CO₂alkyl —CO₂alkyl, —OH, —C(O)-alkyl, —C(O)O-alkyl, —CONH₂, —CO-alkylamino, —CO-dialkylamino, amino, alkylamino, or dialkylamino, any of which may be optionally substituted on carbon with 1, 2, or 3 groups selected from halo, alkyl, OH, or —O-alkyl, which may itself be substituted on carbon with halo;
A is (C₁-C₂₀)alkylene, (C₂-C₂₀)alkenylene, or (C₂-C₂₀)alkynylene, each of which may be optionally substituted on carbon with 1, 2, or 3 groups selected from OH, CO₂H, CO₂alkyl, halogen, amino, alkylamino, dialkylamino, —O-alkyl, alkylene-O-alkyl, alkylene-OH, or alkylene-CO₂H;
X₁ is a bond or is CH₂, O, —CH₂O—, S, —S(O), —S(O)₂, —C(O)—, —C(O)O—, or NRₓ, wherein Rₓ is H or (C₁-C₆)alkyl;
R′ and R″ are each independently hydrogen, halogen, alkyl optionally substituted on carbon with halogen, alkyl, or taken together with the carbon to which they are attached form C═O or a 3, 4, 5, or 6-membered ring, optionally containing 1 or 2 heteroatoms selected from ONH, N-alkyl, SO, or SO₂, any of which may be optionally substituted on carbon with alkyl or halogen;
R₂ₐ is halogen, cyano, alkyl, aryl, heteroaryl, cycloalkyl, heterocycloalkyl, aralkyl, heteroalkyl, —O-alkyl, —O-aryl, —O-heteroaryl, aralkoxy, heteroaralkoxy, —S-alkyl, alkylene—O-alkyl, alkylene-CO₂H, alkylene-CO₂alkyl, alkylSO₂, alkylenesulfonyl, alkylene-CO-amino, alkylene-CO-alkylamino, alkylene-CO-dialkylamino, alkylene-NH—CO₂H, alkylene-NH—CO₂alkyl —CO₂alkyl, —OH, —C(O)-alkyl, —C(O)O-alkyl, —CONH₂, —CO-alkylamino, —CO-dialkylamino, amino, alkylamino, and dialkylamino, any of which may be optionally substituted on carbon with 1, 2, or 3 groups selected from halo, alkyl, OH, or —O-alkyl;
R_{2b} is hydrogen, halogen, cyano, alkyl, aryl, heteroaryl, cycloalkyl, heterocycloalkyl, aralkyl, heteroalkyl, —O-alkyl, —O-aryl, —O-heteroaryl, aralkoxy, heteroaralkoxy, —S-alkyl, alkylene-O-alkyl, alkylene-CO₂H, alkylene-CO₂alkyl, alkylSO₂, alkylenesulfonyl, alkylene-CO-amino, alkylene-CO-alkylamino, alkylene-CO-dialkylamino, alkylene-NH—CO₂H, alkylene-NH—CO₂alkyl —CO₂alkyl, —OH, —C(O)-alkyl, —C(O)O-alkyl, —CONH₂, —CO-alkylamino, —CO-dialkylamino, amino, alkylamino, and dialkylamino, any of which may be optionally substituted on carbon with 1, 2, or 3 groups selected from halo, alkyl, OH, or —O-alkyl;
R₃ is hydrogen, alkyl, or cycloalkyl, either of which may be optionally substituted on carbon with 1, 2, or 3 groups selected from halo, alkyl, OH, or —O-alkyl; 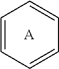
is phenyl or pyridyl;
Y is C₁-C₄ alkylene, optionally substituted on carbon with 1, 2, or 3 groups selected from halo, alkyl, OH, or —O-alkyl,
R₄ is hydrogen;
R₅ and R₆ are each independently selected from the group consisting of hydrogen, alkyl, alkylene-OH, aryl, alkylene-O-alkyl, —CO₂H, CO₂-alkyl, alkylene-OC(O)alkyl, cycloalkyl, heterocyclo, —C(O)-alkyl, —C(O)-aryl, C(O)-aralkyl, —C(O)—Oalkyl, —C(O)—Oaryl, —C(O)—Oaralkyl, alkylene-amino, alkylene-alkylamino, and alkylene-dialkylamino, any of which may be optionally substituted on carbon with halogen, alkyl, hydroxyl, CO₂H, CO₂alkyl or alkoxy; or
R₅ and R₆, together with the nitrogen to which they are attached, may form a 3, 4, 5, or 6-membered saturated or unsaturated ring, optionally containing 1 or 2 additional heteroatoms selected from O, S, NH, or N-alkyl, and optionally substituted on carbon with halogen, alkyl, hydroxyl, or alkoxy; R₅ and R₃ may form a 5, 6, 7, or 8-membered saturated or unsaturated ring, optionally containing 1 or 2 additional heteroatoms selected from O, S, NH, or N-alkyl, and optionally substituted on carbon with halogen, alkyl, hydroxyl, or alkoxy; and
R₇ is selected from the group consisting of hydrogen, alkyl, aryl or aralkyl.

2. A compound of claim 1, wherein R₂ is alkyl substituted with 1, 2 or 3 halo groups.

3. A compound of any of the preceding claims, wherein R₂ is trifluoromethyl.

4. A compound of any of the preceding claims, wherein R₁ is selected from the group consisting of hydrogen, phenyl, pyridyl, thiophenyl, cyclohexyl and cyclopentyl.

5. A compound of any of the preceding claims, wherein A is selected from the group consisting of n-octyl, n-heptyl, n-hexyl, n-pentyl and n-butyl.

6. A compound of any of the preceding claims, wherein X₁ is selected from the group consisting of O, CH₂, C═O and CH₂O.

7. A compound of any of the preceding claims, wherein R₇ is selected from the group consisting of H, methyl, ethyl and benzyl.

8. A compound selected from the group consisting of 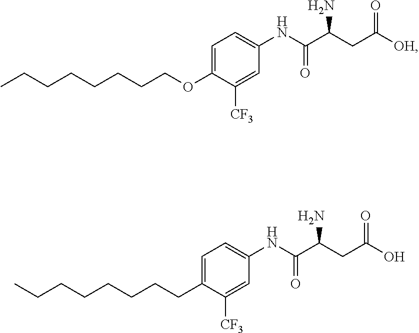
and pharmaceutically acceptable salts, phosphate derivatives, phosphate mimics, or phosphate precursor analogs thereof.

9. A compound as defined in any one of claims 1 to 8 or a pharmaceutically acceptable salt, phosphate derivative, phosphate mimic, or phosphate precursor analog thereof for use as a therapeutic substance.

10. A compound as defined in any one of claims 1 to 8 or a pharmaceutically acceptable salt, phosphate derivative, phosphate mimic, or phosphate precursor analog thereof for use in the treatment of a sphingosine associated disorder.

11. A compound as defined in any one of claims 1 to 8 or a pharmaceutically acceptable salt, phosphate derivative, phosphate mimic, or phosphate precursor analog thereof for use in the treatment of multiple sclerosis.

12. A compound as defined in any one of claims 1 to 8 or a pharmaceutically acceptable salt, phosphate derivative, phosphate mimic, or phosphate precursor analog thereof for use in the manufacture of a medicament for use in the treatment of a sphingosine associated disorder.

13. A compound as defined in any one of claims 1 to 8 or a pharmaceutically acceptable salt, phosphate derivative, phosphate mimic, or phosphate precursor analog thereof for use in the manufacture of a medicament for the treatment of multiple sclerosis.

14. A method of treating a sphingosine 1-phosphate associated disorder comprising administering to a subject a therapeutically effective amount of a compound as defined in any one of claims 1 to 8 or a pharmaceutically acceptable salt, phosphate derivative, phosphate mimic, or a phosphate precursor analog thereof, thereby treating the sphingosine 1-phosphate associated disorder.

15. A pharmaceutical composition comprising a compound as defined in any one of claims 1 to 8 or a pharmaceutically acceptable salt, phosphate derivative, phosphate mimic, or a phosphate precursor analog thereof.

16. A process for the preparation of a pharmaceutical composition according to claim 15.

17. A process for the preparation of a compound as defined in any one of claims 1 to 8 or a pharmaceutically acceptable salt, phosphate derivative, phosphate mimic, or phosphate precursor analog thereof.
